(19) United States
(12) Patent Application Publication
(10) Pub. No.: US 2020/0138399 A1
(43) Pub. Date: May 7, 2020

(54) Wearable stethoscope patch

(71) Applicant: VivaLnk, Inc., Campbell, CA (US)

(72) Inventors: Jiang Li, Cupertino, CA (US); Niel F. Starksen, Los Altos Hills, CA (US); Quoi V. Huynh, Saratoga, CA (US)

(21) Appl. No.: 16/178,843

(22) Filed: Nov. 2, 2018

(51) Int. Cl. A61B 7/04; A61B 5/00; A61B 7/02

(52) U.S. Cl. CPC A61B 7/04; A61B 5/6833; A61B 5/6823; A61B 7/003; A61B 2562/0204; A61B 5/0022; A61B 7/026

(57) ABSTRACT

A stethoscope system includes a wearable stethoscope patch to be worn by a user that includes a substrate, an accelerometer in the substrate, the accelerometer configured to sense acoustic pressure waves in the user's body to produce a first electrical signal, and an antenna over the substrate and in electric communication with the accelerometer, wherein the antenna is configured to wirelessly transmit measurement data based on the first electrical signal. The wearable stethoscope patch also includes a control device that receives the measurement data wirelessly from the antenna and produces stethoscopic data based on the measurement data.

### BACKGROUND OF THE INVENTION

The present application relates to wearable electronic devices, and in particular, to wearable stethoscope that can be worn by a user for picking up acoustic signals of vibration from user's body, such as heart and lung.

Stethoscope is an acoustic medical device for auscultation, or listening to the internal sounds of a user's body. A traditional acoustic stethoscope includes three main mechanical components: a chest piece, tubing, and earpieces. In operation, the chest piece picks up sound from a user's body, an air-filled hollow tube transmits the sound from the chest piece to the earpieces, and then to listener's ears. The chest piece usually consists of two parts that can be placed against the patient for sensing sound: a bell (hollow cup) for picking up lower frequency sounds, and a diaphragm (disc) for picking up higher frequency sounds. When the bell is placed on the patient, the vibrations of the skin directly produce acoustic pressure waves that travel to the listener's ear. When the diaphragm is placed against the patient's skin, body sounds vibrate the diaphragm, creating acoustic pressure waves, which then travel to the listener's ears. These two types of pressure waves allow the listener to examine acoustic vibrations over a wide acoustic frequency range in the user's body.

One drawback associated with the traditional acoustic stethoscopes is that the sound level is normally low. Electronic stethoscopes overcome this problem by electronically amplifying body sounds. Electronic stethoscopes convert acoustic sound waves obtained through the chest piece into electronic signals, which are then processed for optimal listening. The electronic signals can be filtered, digitized, encoded and decoded, to have the ambient noise reduced or eliminated, and sent through speakers or headphones.

An electronic stethoscope typically consists of a microphone, placed in a chest piece, comprising a diaphragm and a transducer. Sound detection can be achieved by placing the chest piece containing the microphone against a user's chest. In some other devices, piezoelectric material is used to convert pressure waves into electrical signals. A piezoelectric crystal can be placed at the head of a metal shaft with the bottom of the shaft making contact with a diaphragm. In another design, piezoelectric crystals are placed within a foam material behind a thick rubber-like diaphragm. In other devices, the sound waves are sensed by a capacitive diaphragm with a conductive inner surface. These methods, however, suffer from ambient noise interferences.

There is therefore still a need for convenient and accurate measurements of acoustic signals in human body.

### SUMMARY OF THE INVENTION

The presently disclosure relates to a wearable stethoscope patch that overcome drawbacks in traditional and conventional technologies. The wearable stethoscope patch can be worn on a person's skin comfortably for a long period of time while a person conducts his or her normal daily activities. The wearable stethoscope patch can accurately and continuously measure and detect acoustic signals in a person's body. The acoustic signals are converted into electrical signals and wirelessly communicated with an external control device. Another advantageous feature of the stethoscope system or wearable stethoscope patch is that it can significantly reduce or remove noise, and thus improve accuracy and usability of the stethoscopic data.

In one general aspect, the present invention relates to a stethoscope system that includes a wearable stethoscope patch adapted to be worn by a user, which includes a substrate, an accelerometer in the substrate, the accelerometer that can sense acoustic pressure waves in the user's body to produce a first electrical signal, and an antenna over the substrate and in electric communication with the accelerometer, wherein the antenna can wirelessly transmit measurement data based on the first electrical signal. The stethoscope system further includes a control device that can receive the measurement data wirelessly from the antenna and to produce stethoscopic data based on the measurement data.

Implementations of the system may include one or more of the following. The control device can include a measurement controller that can transmit a measurement control signal wirelessly to the antenna, wherein the accelerometer can produce the first electrical signal in response to acoustic pressure waves under control of the measurement control signal. The measurement controller can control the accelerometer to vary a type, timing, a frequency, or duration of the first measurement of the user based on the first treatment field applied across the user's body. The wearable stethoscope patch can include multiple accelerometers, each of which can sense acoustic pressure waves in the user's body to produce the first electrical signal, wherein the measurement data can be based on the first electrical signals from the multiple accelerometers. The control device can include a stethoscopic analyzer that can reduce noise in the stethoscopic data by cancelling out uncorrelated signals in the measurement data from the multiple accelerometers. The stethoscopic analyzer can add the measurement data from the multiple accelerometers to cancel out uncorrelated noise signals in the measurement data to reduce noise in the stethoscopic data. The wearable stethoscope patch can include a plurality of circuit modules each comprising a support substrate and a first conductive circuit, wherein at least some of the plurality of circuit modules can include accelerometers on their respective support substrates; and flexible ribbons that connect the plurality of circuit modules, wherein the flexible ribbons and the plurality of circuit modules define one or more openings, wherein the flexible ribbons include second conductive circuits connected to the first conductive circuit. The substrate can include a circuit in electrical communication with the accelerometer and the antenna. The wearable stethoscope patch can further include a semiconductor chip mounted on the substrate and in electrical communication with the circuit. The semiconductor chip can receive the first electrical signal from the accelerometer and convert the first electrical signal to a second electrical signal. The first electrical signal can be analog and the second electrical signal can be digital. The semiconductor chip can enable the antenna to transmit the measurement data based on the second electrical signal.

In another general aspect, the present invention relates to a method for stethoscopic measurement. The method includes sensing acoustic pressure waves in a user's body by an accelerometer in a wearable stethoscope patch attached to the user's body, producing a first electrical signal by the accelerometer in response to acoustic pressure waves, the accelerometer in electric communication with an antenna, wirelessly transmitting measurement data based on the first electrical signal from the antenna to a control device, and producing stethoscopic data by the control device based on the measurement data.

Implementations of the system may include one or more of the following. The method can further include receiving a measurement control signal by the antenna from the control device, and producing the first electrical signal by the accelerometer in response to acoustic pressure waves under control of the measurement control signal. The method can further include sensing acoustic pressure waves in the user's body by multiple accelerometers to produce multiple first electrical signals, wherein the measurement data can be based on the multiple first electrical signals from the multiple accelerometers; and reducing noise in the stethoscopic data by cancelling out uncorrelated signals in the measurement data from the multiple accelerometers. The multiple accelerometers can be mounted on the wearable stethoscope patch. The multiple accelerometers are mounted on a plurality of wearable stethoscope patches. The method can further include amplifying measurement data in a predetermined acoustic spectral range before the step of producing stethoscopic data. The first electrical signal can be analog, wherein the first electrical signal is converted to a second electrical signal by a semiconductor chip, wherein the second electrical signal can be digital, wherein the measurement data is based on the second electrical signal. The method can further include producing an audio signal or a visual display based on the stethoscopic data for diagnostic examination. The wearable stethoscope patch can be attached to a first area of the user's body. The method can further include producing a calibration electrical signal by a calibration accelerometer on a calibration wearable stethoscope patch that is attached to a second area of the user's body away the first area of the user's body; producing calibration data based on the calibration electrical signal; and reducing noise in the stethoscopic data using the calibration data.

These and other aspects, their implementations and other features are described in detail in the drawings, the description and the claims.

### BRIEF DESCRIPTION OF THE DRAWINGS

FIG. 1 illustrates a wearable stethoscope patch attached to a user's body.

FIG. 2 is an exploded cross-sectional view of an exemplified wearable stethoscope patch in accordance with some embodiments of the present invention.

FIG. 3 is an exploded perspective view of another exemplified wearable stethoscope patch in accordance with some embodiments of the present invention.

FIG. 4 is a system block diagram for a control device in wireless communications of the wearable stethoscope patch in accordance with some embodiments of the present invention.

FIG. 5 is a flowchart of using a wearable stethoscope patch to sense acoustic signals from heart and lung in accordance with some embodiments of the present invention.

### DETAILED DESCRIPTION OF THE INVENTION

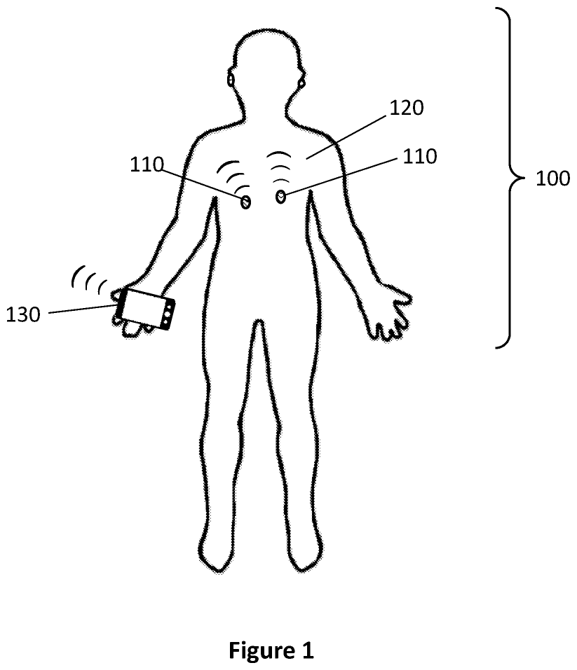

Referring to FIG. 1, a stethoscope system 100 includes one or more wearable stethoscope patches 110 and a control device 130. The one or more wearable stethoscope patches 110 can be attached to the body of a user 120 and sense accelerations or movements in user's skin or tissues caused by acoustic pressure waves in the body of the user 120. The one or more wearable stethoscope patches 110 each can convert the accelerations or movements into electronic signals, and can wirelessly exchange measurement data with the control device 130. The control device 130 can process and analyze measurement data to produce stethoscopic data.

In the present disclosure, the term “wearable patch” can also be referred as “wearable sticker”, “wearable tag”, or “wearable band”, etc. The term “stethoscopic data” refers to internal sounds in human or animal bodies, which can include acoustic (or sound) waves produced in lungs, hearts, intestines, and blood flows in arteries and veins. In some other cases, the vibrations produced by can be directly analyzed to correlate to movement of organs or any physical changes inside the user's body. The acoustic waves produce mechanical vibrations in a user's body comprising internal organs, bones, body fluids, tissues, and the skin.

In the present disclosure, the phrase “acoustic wave” refers to propagating vibrations within the scope of bioacoustics, which includes both human audible and inaudible frequencies. Moreover, “acoustic waves” in the present disclosure can have intensities above and below human hearing discernible levels. The weak acoustic signals from users' bodies can be amplified, and the results can be audibly played or visually displayed for diagnostic examinations.

The control device 130 can be a portable mobile device, which the user 120 can carry with him or her. The control device 130 can also be a stationary device that can be placed at home or office where the user 120 may stay for an extended period. The portable mobile device can be implemented with specialized hardware and software units built in a smart phone, a tablet computer (including devices such as iPod), or a dedicated health or sport monitoring device. The wireless communications can be conducted using Wi-Fi, Bluetooth, Near Field Communication (NFC), and other wireless technologies. The control device 130 can be in communication with a network server in which a user account is stored for the user.

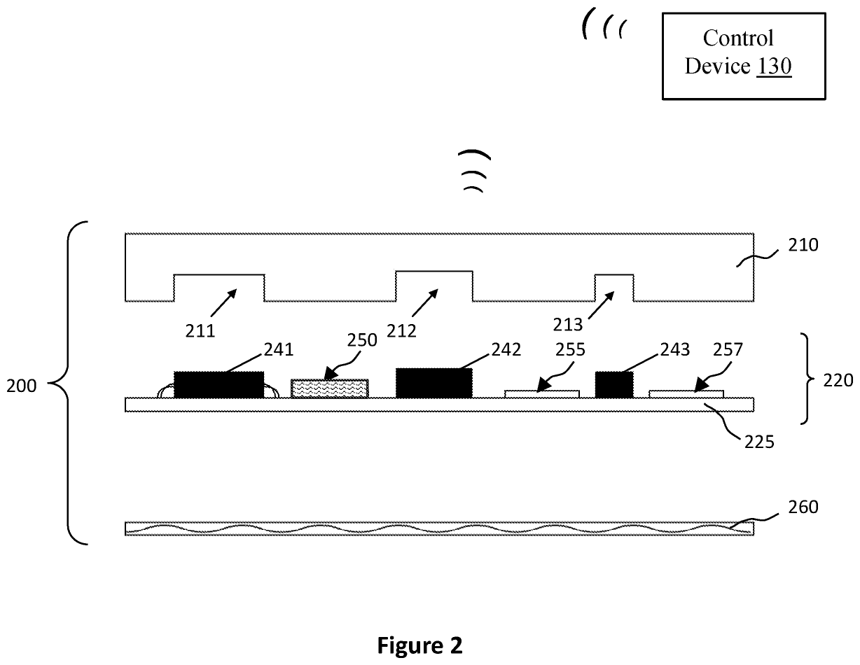

FIG. 2 shows an exemplified wearable stethoscope patch 200 suitable for the stethoscope system 100 in FIG. 1. The wearable stethoscope patch 200 includes an elastic layer 210, a shearable circuit layer 220, and an adhesive layer 260. The shearable circuit layer 220 includes a support substrate 225, a conductive circuit (not shown), semiconductor chips 241-243, an accelerometer 250, electronic components 255, and an antenna 257, which are electrically connected by the conductive circuit.

The electronic components 255 can include a battery, capacitors, inductors, resistors, metal pads, diodes, transistors, amplifiers, etc. The electronic components 255 can also include other sensors for measuring sensors for measuring temperature, blood pressure, voltage, moisture, and pulse measurements, movements, and chemical or biological substances. Through the conductive circuit, the battery powers the semiconductor chips 241-243, the accelerometer 250, other electronic components 255, and the antenna 257.

In some embodiments, the electronic components 255 include electrodes for measuring ECG signals. The electrodes can be formed by electrically conductive pads that are in contact with the user's skin. The ECG signal (voltage) can be measured across two of the electrodes. In particular, the ECG signals can be measured while the stethoscopic measurements are conducted, although the collected ECG signal may be processed differently from stethoscopic information.

The elastic layer 210 can include recesses 211-213 that enclose the semiconductor chips 241-243, which allow the elastic layer 210 to form a substantially flat upper surface. In some embodiments, the elastic layers 210 can be formed on the shearable circuit layer 220 and its associated components thereon by a fluid delivery device such as an ink jet print head, screen printing process, or flexographic process, other layer formation methods known in the art of the field. When the elastic layer 210 is formed on the shearable circuit layer 220 using a fluid delivery device, a polymeric elastic material can be deposited along the contours of the semiconductor chips 241-243, the accelerometer 250, the electronic components 255, and the antenna 257. The elastic layer 210 can be made of soft foam materials such as EVA, PE, CR, PORON, EPD, SCF or fabric textile, to provide stretchability and breathability.

In usage, the adhesive layer 260 is tightly attached the user's check (as shown in FIG. 1) so that the wearable stethoscope patch 200 can pick up acoustic pressure waves associated with sounds in the heart and lung of the user. The accelerometer 250 senses movements created by these acoustic pressure waves and produces a first electrical signal. The accelerometer 250 can be implemented by a micro-electric-mechanical system device (MEMS), wherein the first electrical signal is typical an analog signal. The accelerometer 250 can generate a voltage signal in response to mechanical stress induced by movements, or sense a capacitive change caused by movements. The accelerometer 250 can measure vibrations along one or multiple axes in the bioacoustics range. In some embodiments, the accelerometer 250 can cover the frequency range between 0.2 Hz and 50 KHz for stethoscopic interest.

One of the semiconductor chips 241-243 receives the first electrical signal from the accelerometer 250 via the conductive circuit, and can filter and then digitize the first electrical signal to produce a second digital electrical signal. The first electrical signal and the second digital electrical signal are produced in response to movements created by the acoustic pressure waves, which thus carry information about the sounds in the user's body (e.g. heart and lung).

The semiconductor chips 241-243 can conduct pre-processing of the second digital electrical signal. The semiconductor chips 241-243 produces measurement data based on the second digital electrical signal, and enables the antenna 257 to wirelessly communicate measurement data with the control device 130 (FIG. 1). The wireless signals can be boosted by a wireless boosting station. The wireless signal can be based on using Wi-Fi, Bluetooth, Near Field Communication (NFC), and other wireless standards.

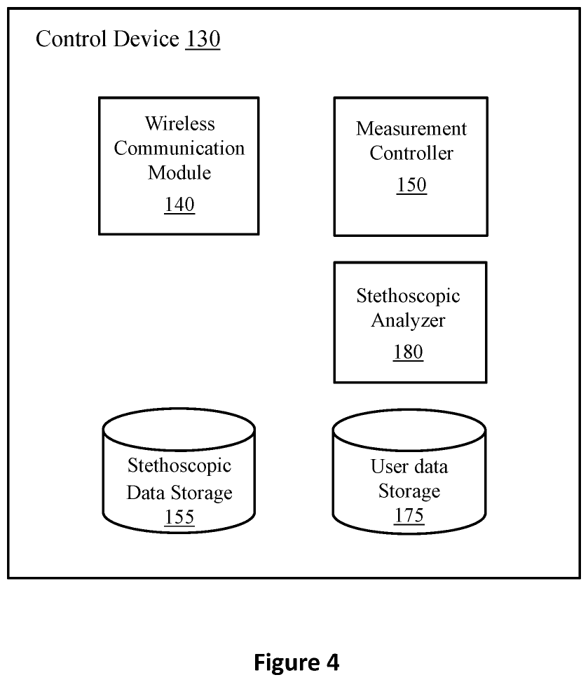

In addition to analog-to-digital conversion and communications, the semiconductor chips 241-243 can also perform logics, signal or data processing, control, calibration, status report, diagnostics, and other functions. In some embodiments, as described below, the semiconductor chips 241-243 can receive measurement control signals from a measurement controller (150 in FIG. 4) to control the measurements conducted by the accelerometer 250 in the wearable stethoscope patch 200.

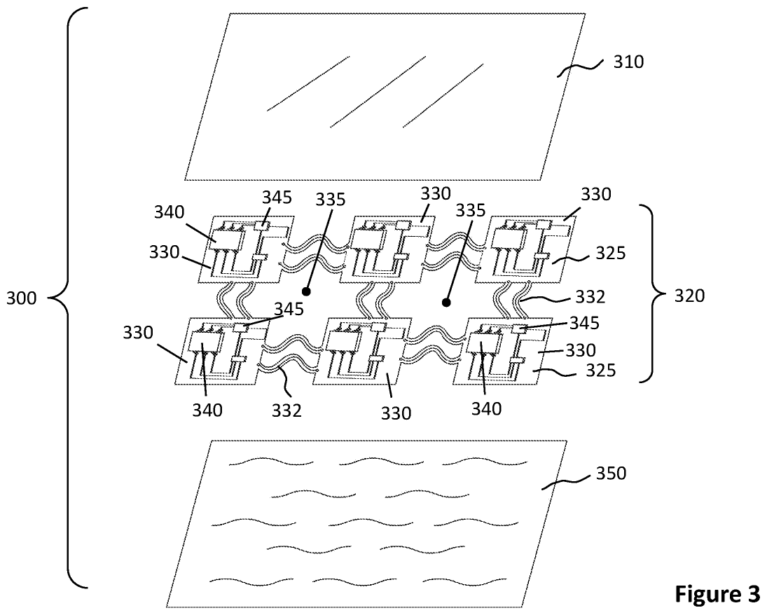

In some embodiments, FIG. 3 shows another exemplified wearable stethoscope patch 300 suitable for the stethoscope system 100 in FIG. 1. The wearable stethoscope patch 300 includes an elastic layer 310, a shearable circuit layer 320, and an adhesive layer 350. The shearable circuit layer 320 includes a network of circuit modules 330 connected by flexible ribbons 332 embedded with conductive lines. The support substrate 325 is flexible and capable of supporting individual IC components in the circuit modules 330. The flexible ribbon 332 can be in curly or serpentine shapes, which allow stretchability when the wearable stethoscope patch 300 is stretched during wearing. As described above, the elastic layers 310 can be made breathable to allow aspiration and moisture from the skin to be released to the environment. The network of individual IC components and/or circuit modules 330 and the flexible ribbons 332 with conductive lines define openings 335 in between to provide additional breathability to the wearable stethoscope patch 300. Furthermore, opening holes or voids can be made on the circuit modules 330 to increase its breathability and the effective elasticity. The support substrate 325 can be contiguous to support the circuit modules 330 and the flexible ribbons 332 with conductive lines. In manufacturing, the support substrate 325 can be formed in a single manufacturing step from a continuous sheet of material. The openings 335 and the connection portions between the circuit modules 330 can be formed by removing material from the continuous sheet by techniques such as laser cutting and/or die cutting. The modules and ribbons in the shearable circuit layer 320 can be made on one single continuous substrate, in which different rigid boards/modules are connected with flexible ribbons via connectors. In the wearable stethoscope patch 300, openings or voids are created on the substrate to provide high effective elasticity and breathability.

At least some circuit modules 330 can each include one or more semiconductor chips 340, an accelerometer 345, and/or electronic components on their respective portions of the support substrates 325, which are connected by a conductive circuit (not shown) within each of the circuit modules 330. The semiconductor chips 340 can perform communications, logic, signal or data processing, control, calibration, status report, diagnostics, and other functions. The electronic components can include an antenna, a battery, capacitors, inductors, resistors, metal pads, diodes, transistors, amplifiers, etc. The electronic components can also include sensors for measuring temperature, blood pressure, voltage, moisture, and pulse measurements, movements, and chemical or biological substances.

In usage, the adhesive layer 350 is tightly attached the user's check (as shown in FIG. 1) so that the wearable stethoscope patch 300 can pick up acoustic pressure waves associated with sounds in the heart and lung of the user. The accelerometer 345 detects the movements created by these acoustic pressure waves and produces a first electrical signal. The accelerometer 345 can be implemented by a micro-electric-mechanical system device (MEMS), wherein the first electrical signal is typical an analog signal. One of the semiconductor chips 340 receives the first electrical signal from the accelerometer 345 via the conductive circuit, and can digitize the first electrical signal to produce a second digital electrical signal. The first electrical signal and the second digital electrical signal are produced in response to movements created by the acoustic pressure waves, which thus carry information about the sounds in the user's body (e.g. heart and lung).

The semiconductor chips 340 can conduct pre-processing of the second digital electrical signal. The semiconductor chips 340 produces measurement data based on the second digital electrical signal, and enables the antenna to wirelessly communicate measurement data with the control device 130 (FIG. 1). The wireless signals can be boosted by a wireless boosting station. The wireless signal can be based on using Wi-Fi, Bluetooth, Near Field Communication (NFC), and other wireless standards.

In addition to analog-to-digital conversion and communications, the semiconductor chips 340 can also perform logics, signal or data processing, control, calibration, status report, diagnostics, and other functions. In some embodiments, as described below, the semiconductor chips 340 can receive measurement control signals from a measurement controller (150 in FIG. 4) to control the measurements conducted by the accelerometer 345 in the wearable stethoscope patch 300.

An advantage of the wearable stethoscope patch 300 is that it includes a plurality of circuit modules 330, which together can hold multiple accelerometers 345 that each can sense movements caused by acoustic pressure waves in the wearing user's body. The flexible ribbons 332 isolate the multiple accelerometers 345 and allow independent measurements of the acoustic waves. As discussed below, such collection of measurements allow noise reduction or cancellation between measurement data obtained from different accelerometers 345, which increase accuracies by the stethoscopic measurements over traditional or conventional techniques.

The control device 130, referring to FIG. 4, includes a wireless communication module 140 that can wirelessly communicate with a wearable stethoscope patch (200, 300 FIGS. 2-3) using above described wireless technologies. The control device 130 includes a measurement controller 150 that controls the wireless communication module 140 to transmit measurement control signals to the wearable stethoscope patches (200, 300 FIGS. 2-3). The measurement controller 150 can vary parameters of the measurements by the wearable stethoscope patches. Such measurement parameters can include types, timing, frequencies, durations of measurements, and coordination between measurements of the same of different accelerometers.

One advantage of the disclosed stethoscope system and wearable stethoscope patches is that stethoscopic measurements can be conducted continuously or in a desired period and at a desired frequency without interfering wearing user's daily activities.

A stethoscopic data storage 155 stores the measurement data obtained by the wearable stethoscope patches (200, 300 FIGS. 2-3). A user data storage 175 stores user data such as user's weight, height, bone density, historic range for blood pressure, heart beat, body temperature, daily patterns of exercises and rests by the user, sickness or symptoms suffered by the user, etc. In some embodiments, as described below, personalized medical treatment can be applied, sometimes dynamically, based on such user data.

A stethoscopic analyzer 180 can process and analyze the measurement data from different wearable stethoscope patches in reference to the user data and the measurement plan (in 150) for the user. The stethoscopic analyzer 180 can pre-store a model that translates user body movements detected by the accelerometers in the wearable stethoscope patches to acoustic pressure values. Using the model, the stethoscopic analyzer 180 generates stethoscopic data based on the measurement data and optionally historic user data. An audio signal can be produced based on the stethoscopic data for diagnostic examination by healthcare personnel. Similarly, a visual display can be produced based on the stethoscopic data for diagnostics. The stethoscopic data can be reported, for example by the wireless communication module 140, to the user, healthcare personnel, or a central server.

As described above, the measurement data can be produced by multiple accelerometers, each of which independently measures movement signal caused by acoustic pressure waves in the wearing user's body. The different accelerometers can be carried by different wearable stethoscope patches (e.g. 110 in FIG. 1). The multiple accelerometers can also be respectively mounted in a same wearable stethoscope patch (e.g. 300 in FIG. 3). The measurement data obtained from different accelerometers can be used to reduce or cancel noise in the measurement data. In one implementation, the measured directional movement data can be added to allow the uncorrelated noise signals to cancel out each other (at least partially), which results in increased accuracies of the stethoscopic data.

In some embodiments, one or more wearable stethoscope patches can be used to calibrate the model and the stethoscopic analyzer 180. For example, the one or more wearable stethoscope patches can be attached to the arms or the legs of a user, away from the user's chest, where the sounds from the lung or the heart of the user are weak. The movements are measured by the accelerometers to produce calibration electrical signals. Calibration data based on the calibration electrical signals are analyzed to obtain patterns in the background noise caused by the ambient environment, the movements of the user, etc. Such noise patterns can be used to remove noise from the stethoscopic data obtained when the wearable stethoscope patches are attached to the chest of a user.

After noise reduction, the measurement data can be amplified by stethoscopic analyzer 180 at specific frequency spectral ranges (e.g. mid-frequency sound) for optimal listening or display.

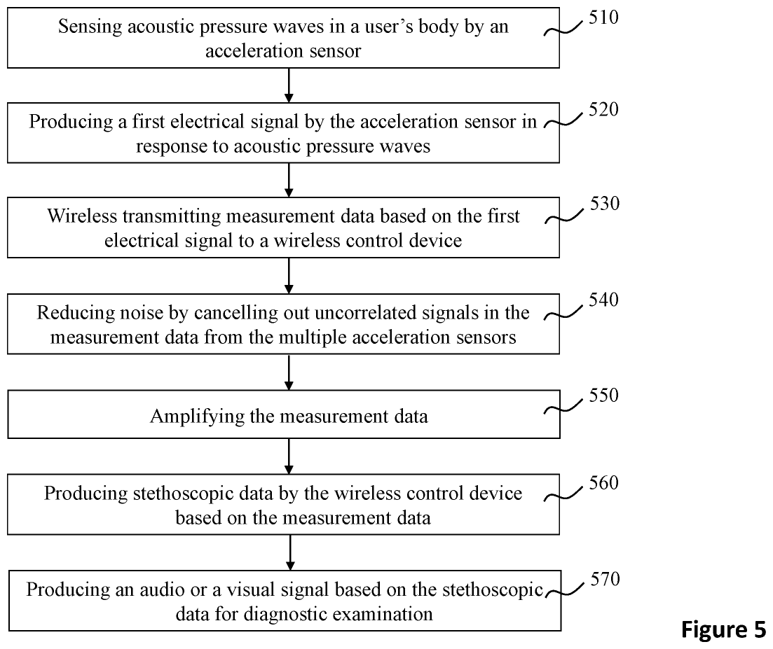

Operation of the disclosed stethoscope system and the disclosed wearable stethoscope patch can include one or more of the following steps. Referring to FIG. 5, acoustic pressure waves in a user's body are sensed by an accelerometer (step 510). A first electrical signal is produced by the accelerometer in response to acoustic pressure waves (step 520), Wireless transmitting measurement data based on the first electrical signal to a control device (step 530). Optionally, noise can be reduced by cancelling out uncorrelated signals in the measurement data from the multiple accelerometers (step 540). Noise can also be reduced by spectral filtering (step 540). The multiple accelerometers can be mounted on the wearable stethoscope patch. Alternatively, the multiple accelerometers can be mounted on a plurality of wearable stethoscope patches. Optionally, the measurement data can be amplified in a predetermined acoustic spectral range (step 550). Stethoscopic data is produced by the control device based on the measurement data (step 560). The wearable stethoscope patch is attached to a first area of the user's body, which is often on the chest or the stomach of the user. An audio signal or a visual display can be produced based on the stethoscopic data for diagnostic examination by healthcare personnel (step 570).

Optionally, stethoscopic measurement by the disclosed stethoscope system and the disclosed wearable stethoscope patch can further include producing a calibration electrical signal by a calibration accelerometer on a calibration wearable stethoscope patch that is attached to a second area of the user's body away the first area of the user's body, producing calibration data based on the calibration electrical signal, and reducing noise in the stethoscopic data using the calibration data.

The disclosed wearable stethoscope patch is stretchable, compliant, durable, and comfortable to wear by users. The disclosed wearable thermometer patch includes a flexible substrate covered and protected by an elastic layer that increases the flexibility and stretchability. Another advantage of the stethoscope system and wearable stethoscope patch is that it can significantly reduce or remove noise, and thus improve accuracy and usability of the stethoscopic data.

While this document contains many specifics, these should not be construed as limitations on the scope of an invention that is claimed or of what may be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination.

Only a few examples and implementations are described. Other implementations, variations, modifications and enhancements to the described examples and implementations may be made without deviating from the spirit of the present invention.

What is claimed is:

1. A stethoscope system, comprising:
a wearable stethoscope patch adapted to be worn by a user, comprising:
a substrate;
an accelerometer in the substrate, the accelerometer configured to sense acoustic pressure waves in the user's body to produce a first electrical signal; and
an antenna over the substrate and in electric communication with the accelerometer, wherein the antenna is configured to wirelessly transmit measurement data based on the first electrical signal; and
a control device configured to wirelessly receive the measurement data from the antenna and to produce stethoscopic data based on the measurement data.

2. The stethoscope system of claim 1, wherein the control device comprises a measurement controller configured to wirelessly transmit a measurement control signal to the antenna, wherein the accelerometer is configured to produce the first electrical signal in response to acoustic pressure waves under control of the measurement control signal.

3. The stethoscope system of claim 2, wherein the measurement controller is configured to control the accelerometer to vary a type, timing, a frequency, or duration of the first measurement of the user based on the first treatment field applied across the user's body.

4. The stethoscope system of claim 1, wherein the wearable stethoscope patch includes multiple accelerometers, each of which configured to sense acoustic pressure waves in the user's body to produce the first electrical signal, wherein the measurement data are based on the first electrical signals from the multiple accelerometers.

5. The stethoscope system of claim 4, wherein the control device includes a stethoscopic analyzer that is configured to reduce noise in the stethoscopic data by cancelling out uncorrelated signals in the measurement data from the multiple accelerometers.

6. The stethoscope system of claim 5, wherein the stethoscopic analyzer is configured to add the measurement data from the multiple accelerometers to cancel out uncorrelated noise signals in the measurement data to reduce noise in the stethoscopic data.

7. The stethoscope system of claim 1, wherein the wearable stethoscope patch comprises:
a plurality of circuit modules each comprising a support substrate and a first conductive circuit, wherein at least some of the plurality of circuit modules comprise accelerometers on their respective support substrates; and
flexible ribbons that connect the plurality of circuit modules, wherein the flexible ribbons and the plurality of circuit modules define one or more openings, wherein the flexible ribbons include second conductive circuits connected to the first conductive circuit.

8. The stethoscope system of claim 1, wherein the substrate includes a circuit in electrical communication with the accelerometer and the antenna, wherein the wearable stethoscope patch further comprises:
a semiconductor chip mounted on the substrate and in electrical communication with the circuit, wherein the semiconductor chip is configured to receive the first electrical signal from the accelerometer and to convert the first electrical signal to a second electrical signal, wherein the first electrical signal is analog and the second electrical signal is digital,
wherein the semiconductor chip is configured to enable the antenna to transmit the measurement data based on the second electrical signal.

9. A stethoscope system, comprising:
a plurality of wearable stethoscope patches adapted to be worn by a user, each comprising:
a substrate;
an accelerometer in the substrate, the accelerometer configured to sense acoustic pressure waves in the user's body to produce a first electrical signal; and
an antenna over the substrate and in electric communication with the accelerometer, wherein the antenna is configured to wirelessly transmit measurement data based on the first electrical signal; and
a control device configured to wirelessly receive the measurement data from the antenna and to produce stethoscopic data based on the measurement data.

10. The stethoscope system of claim 9, wherein the control device includes a stethoscopic analyzer that is configured to reduce noise in the stethoscopic data by cancelling out uncorrelated signals in the measurement data from the plurality of accelerometers on the plurality of wearable stethoscope patches.

11. The stethoscope system of claim 9, wherein the stethoscopic analyzer is configured to add the measurement data from the plurality of accelerometers to cancel out uncorrelated noise signals in the measurement data to reduce noise in the stethoscopic data.

12. A method for stethoscopic measurement, comprising:
sensing acoustic pressure waves in a user's body by an accelerometer in a wearable stethoscope patch attached to the user's body;
producing a first electrical signal by the accelerometer in response to acoustic pressure waves, the accelerometer in electric communication with an antenna;
wirelessly transmitting measurement data based on the first electrical signal from the antenna to a control device; and
producing stethoscopic data by the control device based on the measurement data.

13. The method of claim 12, further comprising:
receiving a measurement control signal by the antenna from the control device; and
producing the first electrical signal by the accelerometer in response to acoustic pressure waves under control of the measurement control signal.

14. The method of claim 12, further comprising:
sensing acoustic pressure waves in the user's body by multiple accelerometers to produce multiple first electrical signals, wherein the measurement data are based on the multiple first electrical signals from the multiple accelerometers; and
reducing noise in the stethoscopic data by cancelling out uncorrelated signals in the measurement data from the multiple accelerometers.

15. The method of claim 14, wherein the multiple accelerometers are mounted on the wearable stethoscope patch.

16. The method of claim 14, wherein the multiple accelerometers are mounted on a plurality of wearable stethoscope patches.

17. The method of claim 12, further comprising:
amplifying measurement data in a predetermined acoustic spectral range before the step of producing stethoscopic data.

18. The method of claim 12, wherein the first electrical signal is analog, wherein the first electrical signal is converted to a second electrical signal by a semiconductor chip, wherein the second electrical signal is digital, wherein the measurement data is based on the second electrical signal.

19. The method of claim 12, further comprising:
producing an audio signal or a visual display based on the stethoscopic data for diagnostic examination.

20. The method of claim 12, wherein the wearable stethoscope patch is attached to a first area of the user's body,
the method further comprising:
producing a calibration electrical signal by a calibration accelerometer on a calibration wearable stethoscope patch that is attached to a second area of the user's body away the first area of the user's body;
producing calibration data based on the calibration electrical signal; and
reducing noise in the stethoscopic data using the calibration data.
